(19) United States
(12) Patent Application Publication
(10) Pub. No.: US 2009/0017517 A1
(43) Pub. Date: Jan. 15, 2009

(54) Preparation of Negative-Stranded RNA Viruses by Electroporation

(75) Inventors: Jeanne Schickli, Sunnyvale, CA (US); Jasmine Kaur, Milpitas, CA (US); Roderick Tang, San Mateo, CA (US)

Correspondence Address: MEDIMMUNE, LLC;Jonathan Klein-Evans, ONE MEDIMMUNE WAY, GAITHERSBURG, MD, 20878, US

(73) Assignee: MedImmune, LLC, Gaithersburg, MD (US)

(21) Appl. No.: 12/171,842

(22) Filed: Jul. 11, 2008

Related U.S. Application Data
(60) Provisional application No. 60/949,728, filed on Jul. 13, 2007.

(51) Int. Cl. C12N 13/00

(52) U.S. Cl. 435/173.6

(57) ABSTRACT

The present invention encompasses methods of preparing non-segmented negative-stranded RNA viruses from cells utilizing electroporation.

### FIELD OF THE INVENTION

The present invention relates to the preparation of negative-stranded RNA viruses by electroporation.

### BACKGROUND OF THE INVENTION

The present invention encompasses methods of preparing negative-stranded RNA viruses from cells following electroporation of nucleic acids into the cells. The methods may be performed in the absence of serum or may be performed in the absence of reagents isolated from an animal. The methods do not require use of a helper virus.

### SUMMARY OF THE INVENTION

The present invention is directed, in part, to serum-free methods of preparing negative-stranded RNA viruses from cells following electroporation of nucleic acids into the cells.

In one aspect, the present invention includes a serum-free method of preparing a non-segmented negative-stranded RNA virus, including, attenuated and/or temperature sensitive non-segmented negative-stranded RNA viruses. The method includes electroporating host cells with nucleic acids including: one or more expression vectors encoding ribonucleoprotein complex proteins of the non-segmented negative-stranded RNA virus, and a vector comprising a nucleotide sequence of a genome or antigenome of the non-segmented negative-stranded RNA virus; and recovering the non-segmented negative-stranded RNA virus.

The method can include that prior to the step of recovering the virus, the electroporated host cells are passaged on expansion cells.

Furthermore, the method can also include culturing the host cells at 32° C. before electroporation. The method can include incubating the electroporated host cells at 32° C. after electroporation. If the method employs expansion cells, the expansion cells may be cultured at 32° C.

The non-segmented negative-stranded RNA virus can be any negative-stranded virus, for example, the virus can be from the family paramyxoviridae. The virus from the family paramyxoviridae may be a subfamily pneumovirinae virus such as respiratory syncytial virus or metapneumovirus. If the virus is from the family paramyxoviridae it may be a subfamily Paramyxovirus virus. If the virus is a subfamily paramyxovirus it may be parainfluenza virus 1, 2, 3, or 4, or may be a Sendai virus, or a Measles virus, or Mumps or Canine distemper virus. If the virus is a respiratory syncytial virus or a human metapneumovirus it may not express an M2-2 gene or may not express another gene.

In one example, the one or more expression vectors and the vector comprising the nucleotide sequence of the genome or antigenome of the non-segmented negative-stranded RNA virus can be plasmids. The plasmids may be endotoxin-free.

The methods encompassed by the invention can also be animal protein-free. In one embodiment of the invention, the one or more expression vectors and the vector including the nucleotide sequence of the genome or antigenome of the non-segmented negative-stranded RNA virus are plasmids prepared from materials that are animal protein-free. In this embodiment, the medium used in the method can be animal protein-free.

The methods encompassed by the invention can further include culturing the electroporated host cells in medium comprising a growth factor. The growth factor can be any growth factor such as an epidermal growth factor, a transforming growth factor alpha, or an insulin-like growth factor.

In another embodiment, expression of the ribonucleoprotein complex proteins is under control of a promoter of an RNA polymerase, such as the T7, SP6 or T3 RNA polymerase, not natively expressed by the host cells, and wherein the host cells are further electroporated with a further expression vector encoding the polymerase such as the T7, SP6 or T3 RNA polymerase, not natively expressed by the host cells.

In the methods encompassed by the invention the total quantity of plasmids used may be between 30 and 55 μg, or between 40 and 50 μg, such as about 46 μg, per approximately 10⁶ to 10⁸ cells. Also, the host cells used in the methods encompassed by the invention can be any host cell including Vero cells.

In another aspect, the invention includes an animal protein-free method of preparing a pneumovirinae virus including electroporating host cells with nucleic acids. The nucleic acids include expression plasmids encoding N, P, and L proteins of the pneumovirinae virus, wherein expression of the N, P, and L proteins of the pneumovirinae virus are under control of a promoter for RNA polymerase, e.g., T7, SP6 or T3 polymerase, an expression plasmid encoding an RNA polymerase, e.g., T7, SP6 or T3 polymerase, and a plasmid comprising a nucleotide sequence of a genome or antigenome of the pneumovirinae virus under control of a promoter for an RNA polymerase such as a T7, SP6 or T3 polymerase, wherein the total quantity of plasmid DNA electroporated in the host cells is between 30 and 55 μg, or between 40 and 50 μg, such as about 46 μg, per approximately 10⁶ to 10⁸ cells; culturing the electroporated host cells on expansion cells; and recovering the pneumovirinae virus from the expansion cells cultured with the electroporated host cells, wherein the plasmids utilized in the step of electroporating are endotoxin-free.

The pneumovirinae virus can be a respiratory syncytial virus or a metapneumovirus. In one embodiment, the pneumovirinae virus is attenuated and/or temperature sensitive. In one embodiment, the host cells are further electroporated with an expression plasmid encoding M2-1 under control of the promoter for RNA polymerase.

The method can further include incubating the electroporated host cells and/or the culture of expansion cells at between 30-40° C., for example, 32° C.

The methods encompassed by the invention can further include culturing the electroporated host cells in medium comprising a growth factor. The growth factor can be any growth factor such as an epidermal growth factor, a transforming growth factor alpha, or an insulin-like growth factor.

In one embodiment, the quantity of plasmid DNA used can be between 30 and 55 μg or between 40 and 50 μg such as about 46 μg. In one example, the molar ratio of expression plasmids encoding the N protein, the P protein, the M2-1 protein, the L protein, the plasmid comprising the nucleotide sequence of the genome or antigenome, and the expression plasmid encoding the RNA polymerase is about 4:4:3:1:1.5:4.

In another embodiment, the host cells are Vero cells. In yet another embodiment, the expansion cells are Vero cells.

In one example, an animal protein-free method of preparing a temperature-sensitive pneumovirus includes: culturing host cells such as Vero cells at a temperature of approximately 32° C.; electroporating the cultured host cells with nucleic acids comprising: expression plasmids encoding N, P, and L proteins of the pneumovirus, wherein expression of the N, P, and L proteins of the pneumovirus are under control of a promoter for T7 polymerase, an expression plasmid encoding T7 polymerase, and a plasmid comprising a nucleotide sequence of a genome or antigenome of the pneumovirus under control of a promoter for polymerase, wherein the total quantity of plasmid DNA electroporated in the host cells is between 30 and 55 μg per approximately 10⁶ to 10⁸ cells; incubating the electroporated host cells at a temperature of approximately 32° C.; transferring the incubated host cells to a culture of expansion cells; and recovering the pneumovirus from the expansion cells cultured with the electroporated host cells, wherein the plasmids utilized in the step of electroporating are endotoxin-free.

The method can further include electroporating the cells with an expression plasmid encoding M2-1 protein of the pneumovirus under control of a promoter for T7 polymerase. In one example, the total quantity of plasmid DNA electroporated in the host cells is about 46 μg. In another example, the molar ratio of expression plasmids encoding the N protein, the P protein, the M2-1 protein, the L protein, the plasmid comprising the nucleotide sequence of the genome or antigenome, and the expression plasmid encoding the T7 polymerase is 4:4:3:1:1.5:4.

### BRIEF DESCRIPTION OF THE FIGURES

FIG. 1 (a)-(c): Schematic genome of (a) RSV and (b) hMPV; (c) characteristics of example RSV and hMPV viruses produced by electroporation in the Examples.

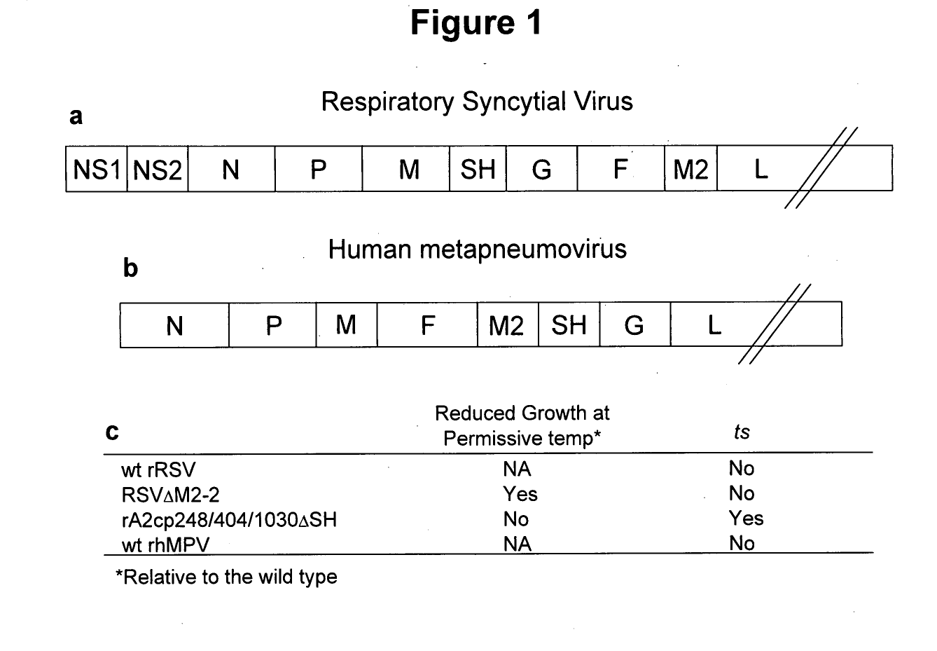

FIG. 2: Photographs of Vero cells grown under varying temperature and serum conditions following electroporation.

FIG. 3: Photographs of Vero cells that had been electroporated to produce rA2 cpts248/404/1030ΔSH grown under varying post-electroporation conditions, e.g., addition of a growth factor or pre-conditioned at 32° C. before electroporation. Photographs in the left column are of cells that had been electroporated and incubated overnight. Photographs in the center column are of cells infected with recovered rA2 cpts248/404/1030 ΔSH at passage 1 that had been immunostained with polyclonal RSV antibody. Photographs in the right column are of cells that had been infected with recovered rA2 cpts248/404/1030ΔSH after 2 plaque expansion steps; areas of syncytia formation indicate virus production.

### DETAILED DESCRIPTION

The present invention encompasses serum-free methods of preparing negative-stranded RNA viruses from cells following electroporation of nucleic acids into the cells. The methods may be performed in the absence of serum or may be performed in the absence of reagents isolated from an animal. The methods do not require use of a helper virus. Moreover because the RNA virus may be recovered via a completely serum-free method and/or animal protein-free method, safety concerns associated with potential introduction of infectious agents that may be present in serum or in animal-derived products is eliminated. Thus, the methods of the present invention provide a means of obtaining a RNA virus that can be used as an immunogenic composition, e.g., as a vaccine virus.

### Electroporation

The invention encompasses methods of preparing negative-stranded RNA viruses that utilize electroporation to introduce nucleic acids into host cells. Electroporation may be conducted using any device known to those of skill in the art. Examples of electroporation devices which are commercially available electroporation include the Gene Pulser Xcell from Bio-Rad, the Multiporator from Eppendorf North America, CelljecT Pro Electroporator from Thermo Scientific, Electroporator 2510 from Eppendorf, MicroPulser electroporator from Bio-Rad, and the EC100 Electroporator from Krackeler.

The electroporation device may be set to any voltage and any capacitance that is capable of introducing nucleic acids into the host cells. For example, the current may be set at a voltage of between 180 and 275 volts, or between 175 and 250 volts, or between 185 and 255 volts, or between 190 and 245 volts, or between 195 and 215 volts, or between 200 and 225 volts, or between 210 and 225 volts, or between 215 and 225 volts. The capacitance may be set between 920 and 980 microfarads, or between 925 and 975 microfarads, or between 930 and 960 microfarads, or between 940 and 960 microfarads, or between 950 and 960 microfarads. The current may be set at between 200 and 230 volts while and capacitance may be set at between 925 and 975 microfarads. The current may be set at approximately 220 volts and the capacitance may be set at approximately 950 microfarads. The current may be set at 220 volts and the capacitance may be set at 950 microfarads.

### Host Cells

The host cells that may be electroporated in the methods encompassed by the invention may be prokaryotic or eukaryotic cells. The host cells may be insect cells such as Sf9 and Sf21, bacterial cells such as E. coli, or yeast cells such as S. cerevisiae. The host cells may be mammalian cells, e.g., human cells. Examples of host cells include human embryonic kidney (HEK) cells, Vero cells (African green monkey kidney cells), WI-38 cells, MRC-5 cells, Cos cells, Fetal Rhesus Lung (FRhL) cells, AGMK-african green monkey kidney cells, human 293 cells, A-549 cells, CHO cells, BHK cells, Madin-Darby Canine Kidney (MDCK) cells, Madin-Darby bovine kidney cells (MDBK), primary chick embryo fibroblast cells, HEp-2 cells, HeLa cells, FIEK (e.g., HEK 293) cells, BHK cells, FRhL-DBS2 cells, LLC-MK2 cells, and primary cells including normal human bronchial epithelium (NHBE) cells and human airway epithelial cells (HAE).

The host cells may be adapted for growth in serum-free medium. A serum-free medium lacks the addition of any serum product, such as fetal bovine serum, fetal calf serum, etc., that is added to a culture medium by either the user or the manufacturer of the medium. The components of the basic serum-free medium include: energy sources such as amino acids, saccharides and organic acids, vitamins, buffer components for pH adjustment, and inorganic salts. Some basic media include MEM, IMDM, Ham's F-12, HyQ SFM4 MegaVir (HyClone) and Williams E. Some serum-free media such as DMEM, VP-SFM and 293SFMII (all from Invitrogen) contain proteins in addition to energy sources such as amino acids, saccharides and organic acids, vitamins, buffer components for pH adjustment, and inorganic salts. Some serum-free media contain proteins that are not of animal origin in addition to energy sources such as amino acids, saccharides and organic acids; vitamins; buffer components for pH adjustment; and inorganic saltsf. Examples of such media include opti-ProSFM. The host cells may not be adapted for growth in serum-free medium. If the host cells are not adapted for growth in serum-free medium they may be capable of growing in serum-free medium for a limited period of time. Alternatively, if the host cells are not adapted for growth in serum-free medium recombinant proteins may be added to the culture medium that allow for survival and replication of the host cells in the absence of serum. Even if the host cells are adapted for growth in serum-free medium recombinant proteins may be added to the culture medium.

Alternatively, the host cells may be grown in an animal protein-free medium. An animal protein-free medium is a serum-free medium that additionally lacks the addition of any animal-derived protein. In one example, the animal protein-free medium lacks an animal derived growth factor. In another example, the animal protein-free medium contains recombinant proteins such as insulin.

Any number of host cells may be electroporated with the nucleic acids. The number of host cells may be, e.g., at least 1, at least 2, at least 5, at least 10, at least 100, at least 1000, at least 10⁴, at least 10⁵, at least 10⁶, at least 10⁷, at least 10⁸, at least 10⁹, or at least 10¹⁰ cells. The host cells, depending upon cell number, can be grown in any type of culture vessel, including plates, dishes, flasks, or bioreactors, and on any scale.

If the host cells are cultured on a small scale, e.g., less than 25 mL medium, the cells may be cultured in culture tubes or flasks. If the host cells are cultured on a larger scale, the cells may be cultured in flasks, in roller bottles, in suspension, or on microcarrier beads (e.g., DEAE-Dextran microcarrier beads, such as Dormacell, Pfeifer & Langen; Superbead, Flow Laboratories; styrene copolymer-tri-methylamine beads, such as Hillex, SoloHill, Ann Arbor). If the host cells are cultured on an even larger scale, the cells may be cultured in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); laboratory and commercial scale bioreactors from B. Braun Biotech International (B. Braun Biotech, Melsungen, Germany). A reactor system may comprise disposable elements such as a flexible plastic bag for culturing cells. Such reactor systems are known in the art and are available commercially. See for example International Patent Publications WO 05/108546; WO 05/104706; and WO 05/10849. Reactor systems comprising disposable elements (also referred herein as “single use bioreactor(s)” or by the abbreviation “SUB(s)”) may be pre-sterilized and do not require a steam-in-place (SIP) or clean-in-place (CIP) environment for changing from batch to batch or product to product in a culture or production system. As such, SUBs require less regulatory control by assuring zero batch-to-batch contamination and can, thus, be operated at a considerable cost-advantage and with minimal or no preparation prior to use. Additionally, since SUBs do not require cleaning or sterilizing, they can be rapidly deployed to facilitate production of large quantities of vaccine material (e.g., virus) from cell culture. In particular embodiments, a disposable reactor system may be a stirred-tank reactor system to allow for a hydrodynamic environment for mixing the cell culture.

The host cells may be cultured at any suitable temperature prior to electroporation. The temperature at which the host cells are cultured may be 37° C., at least 37° C., 38° C., at least 38° C., 39° C., or at least 39° C. Alternatively, the host cells may be cultured at 24° C., at most 24° C., 25° C., at most 25° C., 26° C., at most 26° C., 27° C., at most 27° C., 28° C., at most 28° C., 29° C. at most 29° C., 30° C., at most 30° C., 31° C., at most 31° C., 32° C., at most 32° C., 33° C., at most 33° C., 34° C., at most 34° C., 35° C., at most 35° C., 36° C., at most 36° C., or at a temperature between 37° C. and 39° C., between 30° C. and 34° C., between 32° C. and 34° C., between 28° C. and 30° C., between 34° C. and 38° C., or between 33° C. and 39° C. The host cells may have been culturing at any of these temperatures 2 hours, at least 2 hours, 3 hours, at least 3 hours, 4 hours, at least 4 hours, 5 hours, at least 5 hours, 6 hours, at least 6 hours, 7 hours, at least 7 hours, 8 hours, at least 8 hours, 10 hours, at least 10 hours, 12 hours, at least 12 hours, 18 hours, at least 18 hours, 24 hours, at least 24 hours, 36 hours, at least 36 hours, 4 days, at least 4 days, 5 days, at least 5 days, 7 days, at least 7 days, or between 2 hours and 12 hours, between 4 hours and 24 hours, between 2 hours and 8 hours, or between 4 hours and 8 hours prior to electroporation.

Any of the temperatures at which the host cells may be cultured may be the customary temperature or may not be the customary for culturing the host cells. The host cells may be cultured at a temperature prior to electroporation that is not customary because, e.g., the negative-stranded virus to be produced by the host cells is temperature-sensitive. In such an instance, the host cells may be cultured at a temperature that is lower than the customary temperature prior to electroporation to pre-condition the host cells for the temperature at which the temperature-sensitive virus can replicate. In one embodiment, where the negative-stranded virus is pneumovirus, the host cells may be cultured at a temperature of between 30-34° C.

### Nucleic Acids

Nucleic acids may be electroporated into host cells. Nucleic acids may include one or more expression vector, e.g, that encode ribonucleoprotein complex proteins of the negative-stranded RNA virus, and a vector comprising a nucleotide sequence of a genome or an antigenome of the negative-stranded RNA virus. The one or more expression vector and/or the vector may be any suitable expression vector or vector known in the art. The one or more expression vector and/or vector may be, for example, circularized or linearized plasmids or cosmids or any combination thereof.

The one or more expression vectors encoding the ribonucleoprotein complex proteins may be one expression vector that encodes all ribonucleoprotein complex proteins of a negative-stranded RNA virus, or may be two expression vectors, or at least two expression vectors, three expression vectors, or at least three expression vectors, four expression vectors, or at least four expression vectors.

The one or more expression vectors encoding ribonucleoprotein complex proteins may be under control of a promoter native or not native to the host cells. Examples of mammalian promoters which may be employed include the β-actin promoter, mouse metallothionein-L, human epidermal gene promoter (U.S. Pat. No. 5,643,746), phosphoglycerate kinase promoter, and human or chinese hamster elongation factor 1α promoter. Examples of promoters derived from viral sequences that may be employed include LTR sequences of the Rous sarcoma virus (RSV), Moloney murine leukemia virus (MoMLV), mouse cytomegalovirus (CMV) (Addison et al. J. Gen. Vir. 78 (1997):1653-1661) and human CMV. Further examples of promoters include the early or late Simian Virus 40 promoter, Friend spleen focus-forming virus promoter, and the herpes simplex virus (HSV) thymidine kinase (TK) promoter, the T7 promoter, the T3 promoter, and the SP6 promoter. If the promoter is not native to the host cells and the host cells do not comprise a suitable polymerase that recognizes and controls expression from the promoter, an expression vector encoding a polymerase that recognizes the promoter and drives expression of polypeptides under control of the promoter may further be electroporated into the host cells. For example, an expression vector encoding T7 polymerase may further be electroporated into host cells if expression of the ribonucleoprotein complex proteins is under control of the T7 promoter.

The ribonucleoprotein complex proteins encoded by the one or more expression vectors may include any proteins that encapsidate the RNA genome of the negative-stranded RNA virus. For example, at least the nucleocapsid (N), RNA polymerase (L), and transcription factor (P) encapsidate the RNA genome of negative-stranded RNA viruses. Further proteins, e.g., the M2-1 protein, may encapsidate the viral RNA genome.

The one or more expression vectors, e.g, that encode ribonucleoprotein complex proteins of the negative-stranded RNA virus, and the vector comprising the nucleotide sequence of a genome or an antigenome of a negative-stranded RNA virus may be electroporated into host cells simultaneously. For example, all of the one or more expression vectors and vector may be combined in a single DNA electroporation mixture and simultaneously electroporated into the host cells culture. Alternatively, separate electroporations may be performed for any one or more of the expression vectors or vector. Separate electroporations may be conducted in close temporal sequence to coordinately introduce the expression vectors and vector effectively.

The total quantity of nucleic acids electroporated in the host cells should be of a sufficient quantity that permits virus recovery. One skilled in the art would be readily able to determine the sufficient quantity of nucleic acids and host cells that should be used.

For example, the total quantity of nucleic acids electroporated in the host cells may be between about 35 and 55 μg, may be between about 35 and 50 μg, may be between about 40 and 50 μg, may be between about 40 and 45 μg, may be between about 45 and 50 μg, or may be between about 45 and 47 μg. The total quantity of nucleic acids electroporated in the host cells may be about 40 μg, about 41 μg, about 42 μg, about 43 μg, about 44 μg, about 45 μg, about 46 μg, about 47 μg, about 48 μg, about 49 μg, or about 50 μg per approximately 10⁶ to 10⁸, 10⁶ to 10⁷, or 10⁷ host cells. In one example, between about 35 and 55 μg of total nucleic acids is electroporated in host cells having a cell density of between about 10⁶ to 10⁸.

The nucleic acids used in the methods encompassed by the invention may include expression vectors encoding ribonucleoprotein complex proteins such as N, P, and L of the non-segmented negative-stranded RNA virus, an expression plasmid encoding T7 polymerase and a vector comprising a nucleotide sequence of a genome or antigenome of the non-segmented negative-stranded RNA virus. The nucleic acids employed in the methods may further include an expression vector encoding M2-1.

The quantity of expression vectors encoding the ribonucleoprotein complex proteins, for example the N, P, and L proteins, to be used may be determined relative to the quantity of transcripts that encode the ribonucleoproteins by the native virus. The relative level of transcripts for each of N, P, and L (and optionally M2-1) protein in transfected cells can be controlled by manipulating the molar ratio of plasmids encoding these genes during transfection. For example, during transcription of native RSV DNA, the quantity of transcripts that encode each of these proteins is as follows: N>P>M2>L. The relative level of transcripts for each of N, P, M2-1, and L in transfected cells is therefore controlled by manipulating the molar ratio of plasmids encoding these genes during transfection. In this example, to keep N and P expression at relatively high levels, more plasmids encoding N and P proteins are used than plasmids encoding M2-1. In addition, to avoid excessive expression of the L gene, a lesser amount of plasmid encoding L protein is used relative to the plasmid encoding M2-1 protein.

One skilled in the art would be able to determine the appropriate molar ratio of nucleic acids that should be used based on the RNA virus that is being recovered. In one example, where the RSV virus is being prepared the molar ratio of expression plasmids encoding the N, P, M2-1, and L proteins, the plasmid comprising the nucleotide sequence of the genome or antigenome, and the expression plasmid encoding the T7 polymerase is about 4:4:3:1:1.5:4 The nucleic acids electroporated in the host cells may be prepared by any method known in the art. Such methods are readily available and routinely practiced. See, e.g., Current Protocols in Molecular Cloning Ausubel et al., 1995, John Wiley & Sons, New York; Sambrook et al., 1989, Molecular Cloning: A Laboratory Manual, Second Edition, Cold Spring Harbor Laboratory Press, New York. Commercially available kits that prepare nucleic acids are readily available from, e.g., Qiagen, Stratagene, or Invitrogen. The prepared nucleic acids may or may not be endotoxin-free. Preparation of the nucleic acids that are endotoxin-free can readily be performed, e.g., by the use of commercially available kits that prepare nucleic acids and include reagents for endotoxin removal.

### Virus Recovery

Negative-stranded RNA viruses may be recovered from host cells electroporated with the nucleic acids. Recovery of the negative-stranded RNA viruses may be by simply removing culture medium from cells producing the negative-stranded RNA viruses. Recovery of the negative-stranded RNA viruses may further include a step of purifying the viruses by separating the viruses from cell debris and/or medium components and/or may further include a step of concentrating the virus.

The negative-stranded RNA viruses may be recovered from the electroporated host cells after any period of time sufficient for the host cells to produce the negative-stranded RNA viruses after introduction of the nucleic acids. The negative-stranded RNA viruses may be recovered from the electroporated host cells after at least 6 hours, at least 8 hours, at least 12 hours, at least 18 hours, at least 24 hours, at least 36 hours, at least 48 hours, at least 60 hours, at least 72 hours, at least 3 days, at least 4 days, at least 5 days, at least 6 days, at least 7 days, at least 10 days, at least 12 days, at least 14 days, at least 16 days, at least 18 days, or at least 21 days following introduction of the nucleic acids.

At the time the negative-stranded RNA viruses are recovered from the electroporated host cells the host may be culturing at any temperature that permits the virus' growth and replication. The host cells may be culturing at a temperature of 37° C., e.g., if the viruses are not temperature-sensitive. The host cells may be culturing at a temperature of 37° C., or at least 37° C., 38° C., at least 38° C., 39° C., or at least 39° C. The host cells may be culturing at lower temperatures, e.g., if the viruses are temperature-sensitive. The host cells may be culturing at a temperature of 24° C., at most 24° C., 25° C., at most 25° C., 26° C., at most 26° C., 27° C., at most 27° C., 28° C., at most 28° C., 29° C. at most 29° C., 30° C., at most 30° C., 31° C., at most 31° C., 32° C., at most 32° C., 33° C., at most 33° C., 34° C., at most 34° C., 35° C., at most 35° C., 36° C., or at most 36° C. at the time virus is recovered from the electroporated host cells. Furthermore, the host cells may be culturing at a temperature of between 37° C. and 39° C., between 30° C. and 34° C., between 32° C. and 34° C., between 28° C. and 30° C., 34° C. and 38° C., or between 33° C. and 39° C. at the time virus is recovered from the electroporated host cells.

The host cells may be culturing at the same temperature immediately following electroporation as at the time the virus is recovered. The host cells may be culturing at different temperatures at the time immediately following electroporation and at the time of virus recovery. For instance, at the time immediately following electroporation the host cells may be culturing at a temperature of 35° C., or at least 35° C., 36° C., at least 36° C., 37° C., or at least 37° C., 38° C., at least 38° C., 39° C., or at least 39° C. After culture at this initial temperature the host cells may then be cultured at a different temperature such as 24° C., at most 24° C., 25° C., at most 25° C., 26° C., at most 26° C., 27° C., at most 27° C., 28° C., at most 28° C., 29° C. at most 29° C., 30° C., at most 30° C., 31° C., at most 31° C., 32° C., at most 32° C., 33° C., at most 33° C., 34° C., at most 34° C., 35° C., at most 35° C., 36° C., or at most 36° C. The electroporated host cells may be cultured for approximately 2 hours at the initial temperature and then cultured at the second, different, temperature. The electroporated host cells may be cultured for approximately 2.5 hours, at least 2.5 hours, 3 hours, 3.5 hours, 4 hours, 5 hours, 6 hours, 7 hours, 8 hours, 9 hours, 10 hours, between 2 hours and 10 hours, between 2 hours and 5 hours, between 4 hours and 6 hours, between 4 hours and 7 hours, between 3 hours and 8 hours, between 3 hours and 6 hours, or between 2 hours and 8 hours at the initial temperature before culturing at the second, different, temperature. The host cells may be cultured at more than two different temperatures between the time immediately following electroporation and the time of virus recovery. The host cells may be cultured at three, four, or five different temperatures. The different temperatures may be any of those discussed above.

### Growth Factors

The host cells may be incubated in a medium comprising one or more growth factors following electroporation. The one or more growth factors may be isolated from an animal or an animal tissue, i.e., animal-derived growth factors, or the one or more growth factors may be isolated from a cultured cell line, i.e., animal protein-free growth factors. Any one or more growth factors that maintain host cell growth and/or replication may be present in the culture medium. These growth factors include: epidermal growth factor, fibroblast growth factor, hepatocyte growth factor, insulin-like growth factor, nerve growth factor, platelet-derived growth factor, transforming growth factor alpha, transforming growth factor beta, and/or vascular endothelial growth factor.

The growth factors may be included in the medium of the host cells for any period of time following electroporation. The growth factors may included in the medium of the host cells for 2 hours, at least 2 hours, 2.5 hours, at least 2.5 hours, 3 hours, at least 3 hours, 3.5 hours, 4 hours, at least 4 hours, 5 hours, at least 5 hours, 6 hours, at least 6 hours, 8 hours, at least 8 hours, 10 hours, at least 10 hours, 12 hours, at least 12 hours, 24 hours, at least 24 hours, 36 hours, at least 36 hours, 48 hours, at least 48 hours, 3 days, at least 3 days, 4 days, at least 4 days, 5 days, at least 5 days, 7 days, at least 7 days, 2 weeks, or at least 2 weeks following electroporation. The growth factors may be included in the culture medium of the host cells up until virus recovery.

### Expansion Cells

Following electroporation of cells with the nucleic acids and prior to recovery of the negative-stranded RNA virus, the electroporated host cells, the electroporated host cells and their culture medium, or the culture medium of the electroporated cells may be passaged or cultured on expansion cells in an expansion step. An expansion step can be employed to enrich the quantity of negative-stranded RNA virus ultimately recovered from the electroporated host cells. The successful growth of the virus in the host cells can be determined by any method known in the art such as determining the number of plaques formed. Alternatively, quantitative RT-PCR can be used to determine the amount of virus produced over time, or TCID50 and visualization of CPE (such as syncytia) in which the evidence of CPE in a well with a particular dilution factor is related to the titer of produced virus. Expansion cells include any cells on which the non-segmented negative-stranded RNA virus can replicate. The expansion cells may be the same cells as the host cells or may be of a different cell type. Examples of plaque expansion cells that can be used to support recovery and expansion of recombinant negative-stranded RNA viruses include HEp-2, HeLa, HEK, BHK, FRhL-DBS2, LLC-MK2, MRC-5, A-549, MDBK, and Vero cells.

At the time the electroporated host cells and/or their culture medium is passaged or cultured on the expansion cells, the expansion cells may be present in a monolayer that is at least about 50% confluent, or that is at least about 60% confluent, or is at least about 75% confluent. The surface area of the expansion cells at the time the electroporated cells and/or their culture medium is added may be greater than the surface area used for preparing the virus at the electroporation step.

The expansion step may be performed any period of time following electroporation of the host cells. This period of time may be at least 6 hours, at least 8 hours, 10 hours, at least 10 hours, 12 hours, at least 12 hours, 24 hours, at least 24 hours, 36 hours, at least 36 hours, 48 hours, at least 48 hours, 3 days, at least 3 days, 4 days, at least 4 days, 5 days, at least 5 days, 7 days, at least 7 days, 10 days, at least 10, 2 weeks, or at least 2 weeks, between 12 hours and 1 day, between 1 day and 2 weeks, between 1 day and a week, between 3 days and 5 days, between 3 days and 7 days, between 5 days and 7 days, between 5 days and 10 days, between 7 days and 10 days, between 7 days and 14 days, or between 3 days and 10 days following electroporation.

The period of time of any expansion step, e.g., amount of time expansion cells are incubated with the electroporated cells and/or their media, may be at least 6 hours, at least 8 hours, 10 hours, at least 10 hours, 12 hours, at least 12 hours, 24 hours, at least 24 hours, 36 hours, at least 36 hours, 48 hours, at least 48 hours, 3 days, at least 3 days, 4 days, at least 4 days, 5 days, at least 5 days, 7 days, at least 7 days, 10 days, at least 10, 2 weeks, or at least 2 weeks, between 12 hours and 1 day, between 1 day and 2 weeks, between 1 day and a week, between 3 days and 5 days, between 3 days and 7 days, between 5 days and 7 days, between 5 days and 10 days, between 7 days and 10 days, between 7 days and 14 days, or between 3 days and 10 days.

Virus and/or cells of a first expansion may be further transferred to a second culture of expansion cells, which may be further transferred to a third culture of expansion cells, which may be further transferred to a fourth culture of expansion cells, which may be further transferred to a fifth culture of expansion cells, which may be further transferred to a sixth culture of expansion cells, which may be further transferred to a seventh culture of expansion cells, which may be further transferred to a tenth culture of expansion cells.

Any single expansion step may be conducted for at least 6 hours, at least 8 hours, 10 hours, at least 10 hours, 12 hours, at least 12 hours, 24 hours, at least 24 hours, 36 hours, at least 36 hours, 48 hours, at least 48 hours, 3 days, at least 3 days, 4 days, at least 4 days, 5 days, at least 5 days, 7 days, at least 7 days, 10 days, at least 10, 2 weeks, or at least 2 weeks, between 12 hours and 1 day, between 1 day and 2 weeks, between 1 day and a week, between 3 days and 5 days, between 3 days and 7 days, between 5 days and 7 days, between 5 days and 10 days, between 7 days and 10 days, between 7 days and 14 days, or between 3 days and 10 days. If more than one plaque expansion step is performed it need not be for the same length of time as any other plaque expansion step.

Following any expansion step a non-segmented RNA virus titer of 3 log₁₀, at least 3 log₁₀, 4 log₁₀, at least 4 log₁₀, 5 log₁₀, at least 5 log₁₀, 6 log₁₀, at least 6 log₁₀, 7 log₁₀, at least 7 log₁₀, 8 log₁₀, at least 8 log₁₀, 9 log₁₀, at least 9 log₁₀, between 3 log₁₀ and 9 log₁₀, between 3 log₁₀ and 8 log₁₀, between 4 log₁₀ and 8 log₁₀, between 5 log₁₀ and 8 log₁₀, or between 6 log₁₀ and 8 log₁₀ may be recovered. A titer of between 4 log₁₀ and 8 log₁₀, between 5 log₁₀ and 8 log₁₀, or between 6 log₁₀ and 8 log₁₀ may be recovered after 3 expansion steps. A titer of between 4 log₁₀ and 8 log₁₀, between 5 log₁₀ and 8 log₁₀, or between 6 log₁₀ and 8 log₁₀ may be recovered after 4 plaque expansion steps. A titer of between 4 log₁₀ and 8 log₁₀, between 5 log₁₀ and 8 log₁₀, or between 6 log₁₀ and 8 log₁₀ may be recovered after 5 expansion steps.

### Negative-Stranded RNA Viruses

Any recombinant negative-stranded RNA virus can be recovered using the electroporation procedures discussed herein. Negative-stranded RNA viruses include segmented and non-segmented RNA viruses. Examples of segmented negative-stranded RNA viruses include the orthomyxoviruses, such as influenza virus types A, B, and C, Bunyaviruses such as Hantaviruses, and Arenaviruses. Examples of non-segmented negative-stranded RNA viruses include viruses of the families Paramyxoviridae, Pneumovirinae, Rhabdoviridae and Filoviridae. The family Paramyxoviridae includes subfamilies, Paramyxovirinae and Pneumovirinae. The subfamily Paramyxovirinae includes genera, Respirovirus (formerly Paramyxovirus), Rubulavirus and Morbillivirus. The subfamily Pneumovirinae contains the genus Pneumovirus and the genus Metapneumovirus.

Examples of specific non-segmented negative-stranded RNA viruses that may be prepared by electroporation include Sendai virus (mouse parainfluenza virus type 1), Human parainfluenza virus (PIV) types 1 and 3, Bovine PIV type 3, Simian virus 5 (SV) (Canine parainfluenza virus type 2), Mumps virus, Newcastle disease virus (NDV) (avian Paramyxovirus 1), Human PIV types 2, 4a and 4b, Measles virus (MV), Dolphin Morbillivirus, Canine distemper virus (CDV), Peste-des-petits-ruminants virus, Phocine distemper virus, Rinderpest virus, Hendra virus, Nipah virus, Human respiratory syncytial virus (RSV), Bovine RSV, Pneumonia virus of mice, Human metapneumovirus, Avian pneumovirus (formerly Turkey rhinotracheitis virus), Lyssavirus, Rabies virus, Vesicular stomatitis virus, Bovine ephemeral fever virus, Filovirus, and Marburg virus.

The segmented or non-segmented negative-stranded RNA virus may have an attenuated phenotype or may not have an attenuated phenotype. The segmented or non-segmented negative-stranded RNA virus may be temperature-sensitive or may not be temperature-sensitive. The segmented or non-segmented negative-stranded RNA virus may be infectious and replicating. In general, the segmented or non-segmented negative-stranded RNA virus may be a modified virus that has limited growth potential and/or replication competence and/or infectivity.

If the modified virus has limited growth potential and/or replication competence and/or infectivity it may be limited to only one or two or three or four or five rounds of replication in a host cell or subject. A replication-defective virus may be limited in virus growth, replication and/or infectivity levels that may be at least approximately 10-20%, 20-50%, 50-75%, or 75-95%, or greater than 95% compared to a wild-type or parental virus. If the modified virus is attenuated it may be, e.g., one marked by replication in the lower and/or upper respiratory tract in an accepted animal model (e.g., hamsters, rhesus monkeys or chimpanzees), that is reduced by at least about two-fold, at least about 5-fold, at least about 10-fold, or at least about 20-fold, or about 50-100-fold, or at least 1,000-fold or greater overall (e.g., as measured between 3-8 days following infection) compared to growth of the corresponding wild-type or mutant parental viral strain.

A modified negative-stranded RNA virus may be a virus having amino acid substitutions, nucleotide substitutions, partial or complete gene deletions or knock-outs, e.g., in coding, noncoding, and/or regulatory sequences. A modified negative-stranded RNA virus may have one or more native or heterologous genes that have been selectively altered to manipulate their expression levels, or may have one or more native or heterologous genes that have been added, deleted, or substituted, in whole or in part, alone, or in combination with other modifications. Alternatively, or in addition, the order of genes in the modified virus may be changed, or may have a genome promoter that has been replaced. A modified negative-stranded RNA virus may have modifications in its genomic sequence to facilitate manipulations, such as the insertion of unique restriction sites in various intergenic regions or elsewhere. A modified non-segmented negative-strand RNA virus may have nontranslated gene sequences removed to increase capacity for inserting foreign sequences.

A modified negative-stranded RNA virus comprising a partial or complete gene deletion or gene knock-out can be made according to any methods that are known in the art (as described, for example, in Kretschmer et al., Virology 216:309-316, 1996; Radecke et al., Virology 217:418-421, 1996; Kato et al., EMBO J. 16:578-587, 1987; and Schneider et al., Virology 277:314-322, 1996). A gene knock-out virus can be produced with or without deleting of a gene or genome segment. If the knock-out or gene deletion is produced without deleting a gene or genome segment it may be produced by ablating gene or genome segment expression at the translational or transcriptional level, e.g., by introducing multiple translational termination codons into a translational open reading frame, altering an initiation codon, or modifying an editing site. Example recombinant non-segmented negative-stranded RNA viruses that have a partial or complete gene deletion or gene knock-out include RSV having a partial or complete deletion or knock-out of one or more of the SH, NS1, NS2, and/or M genes or proteins (Jin et al., Virology 273:210-8, 2000). Other example recombinant non-segmented negative-stranded RNA viruses that have a partial or complete gene deletion include VSV with C-terminal deletions affecting the G protein cytoplasmic tail. Further example recombinant non-segmented negative-stranded RNA viruses having partial or complete gene deletions include HPIV having one or more of the C, D, and/or V ORFs ablated or deleted in whole or in part (see, e.g., Durbin et al., Virology 261:319-30, 1999), and MV having all or part of the V protein deleted or knocked out (Schneider et al., Virology 227:314-22, 1997), or mutated (see, e.g., U.S. Pat. No. 6,664,066, issued to Parks on Dec. 16, 2003), or having matrix protein deleted or knocked out (Cathomen et al., EMBO J. 17:3899-908, 1998).

A modified negative-stranded RNA virus comprising one or more rearranged or positionally “shuffled” genes may also be produced by electroporating host cells. Examples of positionally-shifted RSV and VSV viruses are described in, e.g., U.S. Pat. No. 6,596,529, issued to Wertz et al. on Jul. 22, 2003; U.S. Pat. No. 6,136,585, issued to Ball et al. on Oct. 24, 2000; and Wertz et al., Proc. Natl. Acad. Sci. USA 95:3501-6, 1998.

A modified negative-stranded RNA virus comprising partial or complete gene substitutions may also be produced by electroporating host cells. The partial or complete gene substitutions may be substitutions of all or a part of a gene from viruses within the same species but are different isolates, within the same species but are different variants, within the same genus, within the same subfamily, within the same family, within the same order, or may be completely unrelated.

Examples of gene substitutions have been made in HPIV3 are described in, e.g., Durbin et al., Virology 235:323-332, 1997; Skiadopoulos et al., J. Virol. 72:1762-1768, 1998; Tao et al., J Virol 72:2955-2961, 1998; Skiadopoulos et al., J. Virol. 73:1374-1381, 1999; Skiadopoulos et al., Vaccine 18:503-510, 1999; Tao et al., Vaccine 17:1100-1108, 1999; Tao et al., Vaccine 18:1359-1366, 2000; U.S. patent application Ser. No. 09/083,793, filed May 22, 1998; U.S. patent application Ser. No. 09/458,813, filed Dec. 10, 1999; U.S. patent application Ser. No. 09/459,062, filed Dec. 10, 1999; U.S. Provisional Application No. 60/047,575, filed May 23, 1997 (corresponding to International Publication No. WO 98/53078), and U.S. Provisional Application No. 60/059,385, filed Sep. 19, 1997. Such viruses include various host-range restricted human-bovine PIVs (h-bPIV) expressing HPIV or RSV antigenic determinants (Skiadopoulos et al., J. Virol. 77:1141-8, 2003; Bailly et al., J. Virol. 74:3188-95, 2000, Schmidt et al., J. Virol. 75:4594-603, 2001), and RSVs having their F and/or G genes substituted by the F and/or G genes of another RSV, or of VSV (see, e.g., Oomens et al., J. Virol. 77:3785-98, 2003).

A modified negative-stranded RNA virus comprising gene or gene fragment additions may also be produced by electroporating host cells. Such modified viruses may additionally encode, for example, one or more proteins or polypeptides that are antigenic determinants of a pathogen. Examples of antigenic determinants may be polypeptides capable of eliciting an effective immune response against, subgroup A and subgroup B respiratory syncytial viruses (RSV), HMPV, measles virus, human metapneumoviruses, mumps virus, human papilloma viruses, type 1 and type 2 human immunodeficiency viruses, herpes simplex viruses, cytomegalovirus, rabies virus, human metapneuomovirus, Epstein Barr virus, filoviruses, bunyaviruses, flaviviruses, corona viruses (e.g., SARS-associated human corona virus), alphaviruses, or influenza viruses. Examples of such antigens or antigenic determinants that may be added to a modified negative-stranded RNA virus include the measles virus HA and F proteins; the F, G, SH and M2 proteins of RSV, mumps virus HN and F proteins, human papilloma virus L1, L2, E6, or E7 proteins, type 1 or type 2 human immunodeficiency virus gp120 and gp160 proteins, herpes simplex virus and cytomegalovirus gB, gC, gD, gE, gG, gH, gI, gJ, gK, gL, and gM proteins, rabies virus G protein, human metapneuomovirus F and G proteins, Epstein Barr Virus gp350 protein; filovirus G protein, bunyavirus G protein, flavivirus E and NS1 proteins, metapneumovirus G and F proteins, corona virus spike (S) and small membrane (E) proteins, and alphavirus E protein. Antigenic determinants of other pathogens that may be encoded by the modified negative-stranded RNA virus may be those of a viral and bacterial pathogen, or a protozoan or multicellular pathogen. Other antigens are well characterized and known to those of skill in the art.

Alternatively, the modified negative-stranded RNA virus comprising gene or gene fragment additions may additionally encode proteins or polypeptides that modify and/or improve immune response. These proteins or polypeptides include cytokines, for example, an interleukin (IL-2 through IL-18, e.g., interleukin 2 (IL-2), interleukin 4 (IL-4), interleukin 5 (IL-5), interleukin 6 (IL6), interleukin 12 (IL-12), interleukin 18 (IL-18), tumor necrosis factor alpha, interferon gamma, or granulocyte-macrophage colony stimulating factor (GM-CSF) (see, e.g., U.S. application Ser. No. 09/614,285, filed Jul. 12, 2000 and priority U.S. Provisional Application Ser. No. 60/143,425 filed Jul. 13, 1999).

A modified negative-stranded RNA virus which comprises nucleotide alterations may also be produced by electroporating host cells. Such nucleotide alterations may adjust the virus' phenotype, e.g., to increase or decrease attenuation or temperature sensitivity. A nucleotide alteration that confers certain phenotypic characteristics may be well known to those of skill in the art. Examples of known nucleotide alterations that confer certain phenotypic characteristics to non-segmented negative-stranded RNA viruses include those that encode amino acid substitution(s) in the HPIV3 JS cp45 L protein at one or more position corresponding to Tyr942, Leu992, and/or Thr1558. Other examples of known nucleotide alterations that confer certain phenotypic characteristics to non-segmented negative-stranded RNA viruses include those that encode amino acid substitution(s) in RSV L protein corresponding to Phe521. Further nucleotide alterations in a modified non-segmented negative-stranded RNA virus may be introduced in the virus after their initial identification in a heterologous virus as e.g., attenuating, or as conferring temperature sensitivity. Such nucleotide alterations are transferred to the corresponding genomic site in the virus to be modified. This general rational design method for transferring mutations is described in International Application No. PCT/US00/09695, filed Apr. 12, 2000, published as WO 00/61737 on Oct. 19, 2000 corresponding to U.S. National Phase application Ser. No. 09/958,292, filed on Jan. 8, 2002, and claiming priority to U.S. Provisional Patent Application Ser. No. 60/129,006, filed on Apr. 13, 1999, each incorporated herein by reference. Additional description pertaining to such mutations is provided in Newman et al., Virus Genes 24:77-92, 2002; Feller et al., Virology 10; 276:190-201, 2000; Skiadopoulos et al., Virology 260:125-35, 1999; and Durbin et al., Virology 261:319-30, 1999.

Further nucleotide alterations that may be present in modified negative-stranded RNA viruses can be identified by those of skill in the art. Methods of selecting, introducing, and testing various mutations and other modifications of negative-strand RNA viruses are well known and routinely practiced by the skilled artisan. For example, such methods have been described in detail for parainfluenza viruses. See, e.g., Durbin et al., Virology 235:323-332, 1997; U.S. patent application Ser. No. 09/083,793, filed May 22, 1998; U.S. patent application Ser. No. 09/458,813, filed Dec. 10, 1999; U.S. patent application Ser. No. 09/459,062, filed Dec. 10, 1999; U.S. patent application Ser. No. 09/083,793, filed May 22, 1998 (corresponding to International Publication No. WO 98/53078) and its priority U.S. Provisional Applications Nos. 60/047,575, filed May 23, 1997 and 60/059,385, filed Sep. 19, 1997, Newman et al., Virus Genes 24:77-92, 2002, and U.S. Provisional Application No. 60/412,053, filed Sep. 18, 2002. Example methods and materials for cloning respiratory syncytial viruses (RSVs) from cDNA and other disclosure pertaining to selection, introduction, and testing of attenuating mutations and other modifications for use in RSV have been described in, e.g., U.S. patent application Ser. No. 08/720,132, filed Sep. 27, 1996, corresponding to International Publication WO 97/12032 published Apr. 3, 1997, and priority U.S. Provisional Patent Application No. 60/007,083, filed Sep. 27, 1995; U.S. Provisional Patent Application No. 60/021,773, filed Jul. 15, 1996; U.S. Provisional Patent Application No. 60/046,141, filed May 9, 1997; U.S. Provisional Patent Application No. 60/047,634, filed May 23, 1997; U.S. patent application Ser. No. 08/892,403, filed Jul. 15, 1997 corresponding to International Publication No. WO 98/02530 published on Jan. 22, 1998; U.S. patent application Ser. No. 09/291,894, filed on Apr. 13, 1999 corresponding to International Publication No. WO 00/61611 published Oct. 19, 2000, and priority U.S. Provisional Patent Application Ser. Nos. 60/129,006, filed on Apr. 13, 1999; U.S. patent application Ser. No. 09/602,212, filed Jun. 23, 2000 and corresponding International Publication No. WO 01/04335 published on Jan. 18, 2001, and priority U.S. Provisional Patent Application Nos. 60/129,006, filed Apr. 13, 1999, 60/143,097, filed Jul. 9, 1999, and 60/143,132, filed Jul. 9, 1999; International Publication No. WO 00/61737 published on Oct. 19, 2000; Collins et al., Proc Nat. Acad. Sci. U.S.A. 92:11563-11567, 1995; Bukreyev et al., J. Virol. 70:6634-41, 1996, Juhasz et al., J. Virol. 71:5814-5819, 1997; Durbin et al., Virology 235:323-332, 1997; He et al. Virology 237:249-260, 1997; Baron et al. J. Virol. 71:1265-1271, 1997; Whitehead et al., Virology 247:232-9, 1998a; Whitehead et al., J. Virol. 72:4467-4471, 1998b; Jin et al. Virology 251:206-214, 1998; and Whitehead et al., J. Virol. 73:3438-3442, 1999, and Bukreyev et al., Proc. Nat. Acad. Sci. U.S.A. 96:2367-72, 1999.

Growth- or replication-defective non-segmented negative-stranded RNA viruses prepared by electroporation of cells may alternatively, or additionally, have modifications in a leader or trailer region, for example, wherein a trailer region is replaced with a copy of a weaker promoter found in a leader sequence (Finke et al., J. Virol. 73:3818-25, 1999).

A modified non-segmented negative-stranded RNA virus that may be recovered by the methods encompassed by the invention includes any virus disclosed in any one of U.S. patents, applications, patent application publications: U.S. Pat. Nos. 5,882,651, 5,922,326, 6,284,254, 6,264,957, 6,790,449, 5,993,824, 6,689,367, Ser. No. 10/916,827, U.S. Pat. Nos. 6,689,476, 6,713,066, 6,923,971, Ser. Nos. 11/054,343, 11/033,055, U.S. Pat. No. 6,410,023, Ser. Nos. 09/733,692, 10/030,544, 09/900,112 filed Jul. 5, 2001, Ser. No. 10/302,547 filed Nov. 21, 2002, U.S. Pat. No. 6,830,748, Ser. No. 09/724,416 filed Nov. 28, 2000, U.S. Pat. Nos. 6,764,685, 6,811,784, 5,840,520, Ser. No. 11/078,900 filed Mar. 11, 2005, U.S. Pat. No. 6,830,748, Ser. No. 10/672,302 filed Dec. 11, 2006, Ser. No. 10/811,508 filed Mar. 26, 2004, 2005-0118195, 2003-0232061, 2004-0005545, 2003-0232326, 2005-0142148, 2005-0019891, or 2006-0216700.

### Immunogenic Compositions

Negative-stranded RNA viruses produced by electroporation can be used in immunogenic compositions, e.g., vaccines. Such immunogenic compositions may be in liquid form or may be lyophilized and may contain an immunogenically effective amount of the virus.

The immunogenic composition may further contain a physiologically acceptable carrier and/or adjuvant. Useful carriers are well known in the art, and include, e.g., water, buffered water, 0.4% saline, 0.3% glycine, and hyaluronic acid. The resulting immunogenic composition may be packaged for use as an aqueous solution or may be lyophilized. The immunogenic composition may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, or triethanolamine oleate. Acceptable adjuvants include incomplete Freund's adjuvant, MPL™ (3-O-deacylated monophosphoryl lipid A; Corixa, Hamilton Mont.) and IL-12 (Genetics Institute, Cambridge Mass.), among many others well known in the art.

The immunogenic composition may further contain additional viruses to elicit a desired immune response against multiple viral serotypes or strains. Different viruses may be administered in admixture and administered simultaneously or may be administered separately. Immunization with one strain may immunize against different strains of the same or different serotype.

An immunogenic composition may be administered to a subject, e.g., via aerosol, droplet, oral, topical, intramuscular, intranasal, pulmonary, subcutaneous, intravenous, or other route. As a result of administration of the immunogenic composition, the subject may generate an immune response to antigens of the virus, and/or at least partially or completely immune to infection by any virus or other pathogen whose antigens are associated with the virus. Subjects or patients may be animals, e.g., a mammal including a non-primate (e.g., a cow, pig, horse, cat, dog, rat, and mouse) or a primate (e.g., a monkey, such as a cynomolgous monkey, chimpanzee, and a human). The subjects or patients may be mammals, e.g., a human, with a disease or disorder. The subjects or patients may be farm animals (e.g., a horse, pig, or cow) or pets (e.g., a dog or cat) with or without a disorder. The subject may be a mammal (e.g., an immunocompromised or immunosuppressed mammal), at risk of developing a disorder or have one or more symptoms of a disorder.

The quantity of the negative-stranded RNA virus administered to a subject will depend on the subject's state of health and weight, the mode of administration, the nature of the formulation, etc., but will generally range from about 10³ to about 10⁷ plaque forming units (PFU) or more of virus per host, or from about 10⁴ to 10⁶ PFU virus per host. In any event, the immunogenic composition should provide a quantity of non-segmented negative-stranded RNA virus sufficient to elicit a detectable immune response in the subject.

The immunogenic composition may be administered once, or may be administered multiple times. The immunogenic composition may be administered at least two times, at least three times, or at least four times, or more. The immunogenic composition may be administered at a frequency of once a month for one, two, three, four, five or six month, it may be administered at a frequency of once every six months over one or two or three or four intervals, once a year, once every two years, every three years, or every four years.

### Gene Therapy

A non-segmented negative-stranded RNA virus may be modified such that it can be employed as a vector for gene therapy, e.g., of the respiratory tract. If the non-segmented negative-stranded RNA virus is employed as a vector for gene therapy its genome may be altered to encode a gene product of interest by methods well known in the art. Example gene products of interest may include interleukin-2, interleukin-4, gamma-interferon, GM-CSF, G-CSF, erythropoietin, other cytokines, glucocerebrosidase, phenylalanine hydroxylase, cystic fibrosis transmembrane conductance regulator (CFTR), hypoxanthine-guanine phosphoribosyl transferase, cytotoxins, or tumor suppressor genes.

Applicants provide a set of non-limiting embodiments to describe some of the aspects of the invention.

### EMBODIMENT 1

A serum-free method of preparing a non-segmented negative-stranded RNA virus, comprising:
electroporating host cells with nucleic acids comprising:
   a set of one or more expression vectors, said one or more expression vectors encoding ribonucleoprotein complex proteins of the non-segmented negative-stranded RNA virus, and
   a vector comprising a nucleotide sequence of a genome or antigenome of the non-segmented negative-stranded RNA virus; and
recovering the non-segmented negative-stranded RNA virus.

### EMBODIMENT 2

The method of embodiment 1 wherein, prior to the step of recovering, the electroporated host cells and/or media from the electroporated host cells are passaged on expansion cells.

### EMBODIMENT 3

The method of embodiment 1 wherein, the non-segmented negative-stranded RNA virus is attenuated.

### EMBODIMENT 4

The method of embodiment 1 wherein the non-segmented negative-stranded RNA virus is a family paramyxoviridae virus.

### EMBODIMENT 5

The method of embodiment 4 wherein the family paramyxoviridae virus is a subfamily pneumovirinae virus.

### EMBODIMENT 6

The method of embodiment 5 wherein the subfamily pneumovirinae virus is respiratory syncytial virus.

### EMBODIMENT 7

The method of embodiment 6 wherein the respiratory syncytial virus does not express a M2-2 gene.

### EMBODIMENT 8

The method of embodiment 4 wherein the family paramyxoviridae virus is parainfluenza virus.

### EMBODIMENT 9

The method of embodiment 5 wherein the subfamily pneumovirinae virus is metapneumovirus.

### EMBODIMENT 10

The method of embodiment 1 wherein the one or more vectors of the set of expression vectors and the vector comprising the nucleotide sequence of the genome or antigenome of the non-segmented negative-stranded RNA virus are endotoxin free.

### EMBODIMENT 11

The method of embodiment 1 or embodiment 10 which is further animal protein-free.

### EMBODIMENT 12

The method of any one of embodiments 1, 6, 7, 8, or 9 wherein the one or more vectors of the set of expression vectors and the vector comprising the nucleotide sequence of the genome or antigenome of the non-segmented negative-stranded RNA virus are plasmids.

### EMBODIMENT 13

The method of embodiment 12 wherein the plasmids are endotoxin-free.

### EMBODIMENT 14

The method of embodiment 13 which is further animal protein-free.

### EMBODIMENT 15

The method of embodiment 12 wherein:
expression of the ribonucleoprotein complex proteins is under control of a promoter of a polymerase not natively expressed by the host cells, and

wherein the host cells are further electroporated with a further expression vector encoding the polymerase not natively expressed by the host cells.

### EMBODIMENT 16

The method of embodiment 13 wherein:
expression of the ribonucleoprotein complex proteins is under control of a promoter of a polymerase not natively expressed by the host cells, and
wherein the host cells are further electroporated with a further expression vector encoding the polymerase not natively expressed by the host cells.

### EMBODIMENT 17

The method of embodiment 14 wherein:
expression of the ribonucleoprotein complex proteins is under control of a promoter of a polymerase not natively expressed by the host cells, and
wherein the host cells are further electroporated with a further expression vector encoding the polymerase not natively expressed by the host cells.

### EMBODIMENT 18

The method of embodiment 15 wherein the polymerase is T7 polymerase.

### EMBODIMENT 19

The method of embodiment 16 wherein the polymerase is T7 polymerase.

### EMBODIMENT 20

The method of embodiment 17 wherein the polymerase is T7 polymerase.

### EMBODIMENT 21

The method of embodiment 12 wherein the total quantity of plasmids is between about 40 and 50 μg.

### EMBODIMENT 22

The method of embodiment 13 wherein the total quantity of plasmids is between about 40 and 50 μg.

### EMBODIMENT 23

The method of embodiment 14 wherein the total quantity of plasmids is between about 40 and 50 μg.

### EMBODIMENT 24

The method of embodiment 15 wherein the total quantity of plasmids is between about 40 and 50 μg.

### EMBODIMENT 25

The method of embodiment 16 wherein the total quantity of plasmids is between about 40 and 50 μg.

### EMBODIMENT 26

The method of embodiment 17 wherein the total quantity of plasmids is between about 40 and 50 μg.

### EMBODIMENT 27

The method of embodiment 21 wherein the total quantity of plasmids is about 46 μg.

### EMBODIMENT 28

The method of embodiment 22 wherein the total quantity of plasmids is about 46 μg.

### EMBODIMENT 29

The method of embodiment 23 wherein the total quantity of plasmids is about 46 μg.

### EMBODIMENT 30

The method of embodiment 24 wherein the total quantity of plasmids is about 46 μg.

### EMBODIMENT 31

The method of embodiment 25 wherein the total quantity of plasmids is about 46 μg.

### EMBODIMENT 32

The method of embodiment 26 wherein the total quantity of plasmids is about 46 μg.

### EMBODIMENT 33

The method of any one of embodiments 1, 4, 6, 7, 8, or 9 wherein the host cells are Vero cells.

### EMBODIMENT 34

An animal protein-free method of preparing a pneumovirinae virus comprising:
electroporating host cells with nucleic acids comprising:
   expression plasmids encoding N, P, and L proteins of the pneumovirinae virus, wherein expression of the N, P, and L proteins of the pneumovirinae virus are under control of a promoter for T7 polymerase,
   an expression plasmid encoding T7 polymerase, and
   a plasmid comprising a nucleotide sequence of a genome or antigenome of the pneumovirinae virus,
wherein the total quantity of plasmid DNA electroporated in the host cells is between about 40 and 50 μg, and
wherein electroporation is performed by applying one pulse of current at 220 V and 950 microfarads,
culturing the electroporated host cells on expansion cells; and
recovering the pneumovirinae virus from the expansion cells cultured with the electroporated host cells;

wherein the plasmids utilized in the step of electroporating are endotoxin-free.

### EMBODIMENT 35

The method of embodiment 34 wherein the pneumovirinae virus is a respiratory syncytial virus.

### EMBODIMENT 36

The method of embodiment 34 wherein the pneumovirinae virus is a metapneumovirus.

### EMBODIMENT 37

The method of embodiment 35 or embodiment 36 wherein the host cells are further electroporated with an expression plasmid encoding M2-1 under control of the promoter for T7 polymerase.

### EMBODIMENT 38

The method of embodiment 37 wherein the total quantity of plasmid DNA electroporated in the host cells is about 46 μg.

### EMBODIMENT 39

The method of embodiment 37 wherein the host cells are Vero cells.

### EMBODIMENT 40

The method of embodiment 39 wherein the expansion cells are Vero cells.

### EMBODIMENT 41

The method of embodiment 37 wherein the molar ratio of expression plasmids encoding the N protein, the P protein, the M2-1 protein, the L protein, the plasmid comprising the nucleotide sequence of the genome or antigenome, and the expression plasmid encoding the T7 polymerase is approximately 4:4:3:1:1.5:4.

### EMBODIMENT 42

A serum-free method of preparing a non-segmented negative-stranded RNA virus comprising:
electroporating host cells with nucleic acids comprising:
   a set of one or more expression vectors, said one or more expression vectors encoding ribonucleoprotein complex proteins of the non-segmented negative-stranded RNA virus, and
   a vector comprising a nucleotide sequence of a genome or antigenome of the non-segmented negative-stranded RNA virus;
culturing the electroporated host cells in medium comprising a growth factor; and
recovering the non-segmented negative-stranded RNA virus.

### EMBODIMENT 43

The method of embodiment 42 wherein the one or more expression vectors and the vector comprising the nucleotide sequence of the genome or antigenome of the non-segmented negative-stranded RNA virus are plasmids.

### EMBODIMENT 44

The method of embodiment 43 wherein the expression of the ribonucleoprotein complex proteins is under control of a promoter of a polymerase not natively expressed by the host cells, and

wherein the host cells are further electroporated with a further expression vector encoding the polymerase not natively expressed by the host cells.

### EMBODIMENT 45

The method of embodiment 44 wherein the polymerase not natively expressed by the host cells is T7 polymerase.

### EMBODIMENT 46

The method of embodiment 44 or embodiment 45 wherein the plasmids are endotoxin-free.

### EMBODIMENT 47

The method of embodiment 46 wherein the total quantity of the plasmids is between about 40 and 50 μg.

### EMBODIMENT 48

The method of embodiment 47 wherein the total quantity of the plasmids is about 46 μg.

### EMBODIMENT 49

The method of embodiment 42, 43, or 45 wherein the host cells are Vero cells.

### EMBODIMENT 50

The method of embodiment 46 wherein the host cells are Vero cells.

### EMBODIMENT 51

The method of embodiment 49 wherein, prior to the step of recovering, the electroporated host cells and/or culture media of the electroporated host cells are passaged on expansion cells.

### EMBODIMENT 52

The method of embodiment 51 wherein the expansion cells are Vero cells.

### EMBODIMENT 53

The method of any one of embodiments 42, 43, 44, or 45 which is further animal protein-free.

### EMBODIMENT 54

The method of embodiment 49 which is further animal protein-free.

### EMBODIMENT 55

The method of embodiment 52 which is further animal protein-free.

### EMBODIMENT 56

The method of embodiment 53 wherein the growth factor is one of epidermal growth factor, transforming growth factor alpha, or insulin-like growth factor.

### EMBODIMENT 57

The method of embodiment 54 wherein the growth factor is one of epidermal growth factor, transforming growth factor alpha, or insulin-like growth factor.

### EMBODIMENT 58

The method of embodiment 55 wherein the growth factor is one of epidermal growth factor, transforming growth factor alpha, or insulin-like growth factor.

### EMBODIMENT 59

The method of embodiment 53 wherein the non-segmented negative-stranded RNA virus is a family paramyxoviridae virus.

### EMBODIMENT 60

The method of embodiment 56 wherein the non-segmented negative-stranded RNA virus is a family paramyxoviridae virus.

### EMBODIMENT 61

The method of embodiment 59 wherein the family paramyxoviridae virus is a subfamily pneumovirinae virus.

### EMBODIMENT 62

The method of embodiment 60 wherein the family paramyxoviridae virus is a subfamily pneumovirinae virus.

### EMBODIMENT 63

The method of embodiment 61 wherein the subfamily pneumovirinae virus is a respiratory syncytial virus.

### EMBODIMENT 64

The method of embodiment 62 wherein the subfamily pneumovirinae virus is a respiratory syncytial virus.

### EMBODIMENT 65

The method of embodiment 61 wherein the subfamily pneumovirinae virus is a metapneumovirus.

### EMBODIMENT 66

The method of embodiment 62 wherein the subfamily pneumovirinae virus is a metapneumovirus.

### EMBODIMENT 67

The method of embodiment 42 wherein the non-segmented negative-stranded RNA virus is attenuated.

### EMBODIMENT 68

The method of embodiment 42 wherein the non-segmented negative-stranded RNA virus is temperature-sensitive.

### EMBODIMENT 69

An animal protein-free method of preparing a pneumovirus comprising:
electroporating host cells with nucleic acids comprising:
   expression plasmids encoding N, P, and L proteins of the pneumovirus, wherein expression of the N, P, and L proteins of the pneumovirus are under control of a promoter for T7 polymerase,
   an expression plasmid encoding T7 polymerase, and
   a plasmid comprising a nucleotide sequence of a genome or antigenome of the pneumovirus,
wherein the total quantity of plasmid DNA electroporated in the host cells is between about 40 and 50 μg, and
wherein electroporation is performed by applying one pulse of current at about 220 V and 950 microfarads,
incubating the electroporated cells in medium comprising a growth factor selected from the group consisting of epidermal growth factor, human transforming growth factor alpha, and insulin-like growth factor;
transferring the electroporated host cells to a culture of expansion cells; and
recovering the pneumovirus from the expansion cells cultured with the electroporated host cells;

wherein the plasmids utilized in the step of electroporating are endotoxin-free.

### EMBODIMENT 70

The method of embodiment 69 wherein the pneumovirus is attenuated.

### EMBODIMENT 71

The method of embodiment 69 wherein the pneumovirus is temperature sensitive.

### EMBODIMENT 72

The method of embodiment 70 wherein the pneumovirus is a respiratory syncytial virus.

### EMBODIMENT 73

The method of embodiment 71 wherein the pneumovirus is a respiratory syncytial virus.

### EMBODIMENT 74

The method of embodiment 70 wherein the pneumovirus is a metapneumovirus.

### EMBODIMENT 75

The method of embodiment 71 wherein the pneumovirus is a metapneumovirus.

### EMBODIMENT 76

The method of embodiment 71 wherein the electroporated host cells and the electroporated host cells transferred to the culture of expansion cells are incubated at 32° C.

### EMBODIMENT 77

The method of embodiment 69 wherein the host cells are Vero cells.

### EMBODIMENT 78

The method of embodiment 77 wherein the expansion cells are Vero cells.

### EMBODIMENT 79

The method of embodiment 69 wherein total quantity of plasmid DNA electroporated in the host cells is about 46 μg.

### EMBODIMENT 80

The method of embodiment 79 wherein the host cells are further electroporated with an expression plasmid encoding M2-1 under control of the promoter for T7 polymerase.

### EMBODIMENT 81

The method of embodiment 80 wherein the molar ratio of expression plasmids encoding the N protein, the P protein, the M2-1 protein, the L protein, the plasmid comprising the nucleotide sequence of the genome or antigenome, and the expression plasmid encoding the T7 polymerase is about 4:4:3:1:1.5:4.

### EMBODIMENT 82

A serum-free method of preparing a temperature-sensitive non-segmented negative-stranded RNA virus comprising:
culturing host cells in a medium at a temperature of approximately 32° C.;
electroporating the cultured host cells with nucleic acids comprising:
   a set of one or more expression vectors, said one or more expression vectors encoding ribonucleoprotein complex proteins of the non-segmented negative-stranded RNA virus, and
   a vector comprising a nucleotide sequence of a genome or antigenome of the non-segmented negative-stranded RNA virus;
incubating the electroporated host cells at a temperature of approximately 32° C.;
recovering the non-segmented negative-stranded RNA virus.

### EMBODIMENT 83

The method of embodiment 82 which is further animal protein-free.

### EMBODIMENT 84

The method of embodiment 82 wherein the one or more expression vectors and the vector comprising the nucleotide sequence of the genome or antigenome of the non-segmented negative-stranded RNA virus are plasmids.

### EMBODIMENT 85

The method of embodiment 84 wherein the plasmids are endotoxin-free.

### EMBODIMENT 86

The method of embodiment 85 wherein the total quantity of the plasmids is between about 40 and 50 μg.

### EMBODIMENT 87

The method of embodiment 86 wherein the total quantity of the plasmids is about 46 μg.

### EMBODIMENT 88

The method of embodiment 82 or embodiment 85 wherein the expression of the ribonucleoprotein complex proteins is under control of a promoter of a polymerase not natively expressed by the host cells, and wherein the host cells are further electroporated with a further expression vector encoding the polymerase not natively expressed by the host cells.

### EMBODIMENT 89

The method of embodiment 88 wherein the polymerase not natively expressed by the host is T7 polymerase.

### EMBODIMENT 90

The method of embodiment 82 wherein the host cells are Vero cells.

### EMBODIMENT 91

The method of embodiment 88 wherein the host cells are Vero cells.

### EMBODIMENT 92

The method of embodiment 82 or embodiment 90 wherein, prior to the step of recovering, the electroporated host cells are transferred on expansion cells.

### EMBODIMENT 93

The method of embodiment 91 wherein prior to the step of recovering, the electroporated host cells are transferred on expansion cells.

### EMBODIMENT 94

The method of embodiment 92 wherein the expansion cells are Vero cells.

### EMBODIMENT 95

The method of embodiment 93 wherein the expansion cells are Vero cells.

### EMBODIMENT 96

The method of embodiment 81 wherein the non-segmented negative-stranded RNA virus is family paramyxoviridae virus.

### EMBODIMENT 97

The method of embodiment 96 wherein the family paramyxoviridae virus is a subfamily pneumovirinae virus.

### EMBODIMENT 98

The method of embodiment 97 wherein the subfamily pneumovirinae virus is a respiratory syncytial virus.

### EMBODIMENT 99

The method of embodiment 97 wherein the subfamily pneumovirinae virus is a metapneumovirus.

### EMBODIMENT 100

The method of embodiment 95 wherein the non-segmented negative-stranded RNA virus is family paramyxoviridae virus.

### EMBODIMENT 101

The method of embodiment 100 wherein the family paramyxoviridae virus is a subfamily pneumovirinae virus.

### EMBODIMENT 102

The method of embodiment 101 wherein the subfamily pneumovirinae virus is a respiratory syncytial virus.

### EMBODIMENT 103

The method of embodiment 101 wherein the subfamily pneumovirinae virus is a metapneumovirus.

### EMBODIMENT 104

An animal protein-free method of preparing a temperature-sensitive pneumovirinae virus comprising:
culturing host cells at a temperature of approximately 32° C.;
electroporating the cultured host cells with nucleic acids comprising:
   expression plasmids encoding N, P, and L proteins of the pneumovirinae virus, wherein expression of the N, P, and L proteins of the pneumovirinae virus are under control of a promoter for T7 polymerase,
   an expression plasmid encoding T7 polymerase, and
   a plasmid comprising a nucleotide sequence of a genome or antigenome of the pneumovirinae virus,
wherein the total quantity of plasmid DNA electroporated in the host cells is between about 40 and 50 μg, and
wherein electroporation is performed by applying one pulse of current at 220 V and 950 microfarads;
incubating the electroporated host cells at a temperature of approximately 32° C.;
transferring the incubated host cells to a culture of expansion cells; and
recovering the pneumovirinae virus from the expansion cells cultured with the electroporated host cells;

wherein the plasmids utilized in the step of electroporating are endotoxin-free.

### EMBODIMENT 105

The method of embodiment 104 wherein the cultured host cells are further electroporated with an expression plasmid encoding M2-1 protein of the pneumovirinae virus under control of a promoter for T7 polymerase.

### EMBODIMENT 106

The method of embodiment 105 wherein the host cells are Vero cells.

### EMBODIMENT 107

The method of embodiment 105 wherein the expansion cells are Vero cells.

### EMBODIMENT 108

The method of embodiment 106 wherein the expansion cells are Vero cells.

### EMBODIMENT 109

The method of embodiment 104 wherein the total quantity of plasmid DNA electroporated in the host cells is about 46 μg.

### EMBODIMENT 110

The method of embodiment 108 or embodiment 109 wherein the pneumovirinae virus is a respiratory syncytial virus.

### EMBODIMENT 111

The method of embodiment 108 or 109 wherein the pneumovirinae virus is a metapneumovirus.

### EMBODIMENT 112

The method of embodiment 105 wherein the pneumovirinae virus is a respiratory syncytial virus or a metapneumovirus.

### EMBODIMENT 113

The method of embodiment 112 wherein the total quantity of plasmid DNA electroporated in the host cells is about 46 μg.

### EMBODIMENT 114

The method of embodiment 113 wherein the molar ratio of expression plasmids encoding the N protein, the P protein, the M2-1 protein, the L protein, the plasmid comprising the nucleotide sequence of the genome or antigenome, and the expression plasmid encoding the T7 polymerase is about 4:4:3:1:1.5:4.

### EMBODIMENT 115

The method of embodiment 104 wherein the non-segmented negative-stranded RNA virus is further attenuated.

This application claims priority to application Ser. No. 60/949,728 filed Jul. 13, 007, incorporated by reference in its entirety for all purposes.

All publications, patents and patent applications mentioned in this specification are herein incorporated by reference into the specification in their entirety.

The set of examples that follow are provided for the purpose of illustration only and the invention should in no way be construed as being limited to these examples.

### EXAMPLES

### Example 1

### Selection of Ratio of Plasmids for RSV Rescue by Electroporation

To prepare RSV from DNA, nucleotide sequences encoding nucleoprotein (N), phosphoprotein (P), M2-1 protein, which influences processivity, and polymerase (L) would be transfected into cells with antigenomic DNA to produce functional viral RNP complexes in the cells. In the RSV RNA genome, N is most proximal to the leader sequence while P, M2 and L are consecutively more distal from the leader. Therefore, during transcription of native RSV DNA, the quantity of transcripts that encode each of these proteins is as follows: N>P>M2>L. The relative level of transcripts for each of N, P, M2-1, and L in transfected cells was controlled by manipulating the molar ratio of plasmids encoding these genes during transfection. For example, to keep N and P expression at relatively high levels, more plasmids encoding N and P proteins were used than plasmids encoding M2-1. In addition, to avoid excessive expression of the L gene, a lesser amount of plasmid encoding L protein was used relative to the plasmid encoding M2-1 protein.

The plasmids encoding RSV N, RSV P and RSV M2-1 have nucleotide lengths of between 4 kb to 5 kb. The plasmid encoding RSV L is 10 kb. The plasmid containing the RSV antigenome is roughly 18 kb. A plasmid encoding T7 RNA polymerase (7 kb) was also included in the transfection; T7 RNA polymerase is required to drive expression of the RSV plasmids. The ratio, by weight, of plasmids encoding either N, P, M2-1, L, full-length RSV and T7 was 8 μg, 8 μg, 6 μg, 4 μg, 10 μg, and 10 μg, respectively. These weights roughly corresponded to a molar ratio of 4, 4, 3, 1, 1.5 and 4 for plasmids pCITE RSV N, pCITE RSV P, pCITE RSV M2-1, pCITE RSV L, full-length RSV antigenome, and pADT7(N)ΔpT7, respectively. This ratio was maintained for recovery of all viruses and all conditions studied in the remaining Examples.

### Example 2

### Total Quantity of DNA For Rescuing RSV by Electroporation

RSV vaccine candidate rRSVΔM2-2 was used as a test virus to determine the total quantity of DNA that should be used for virus production using electroporation. rRSVΔM2-2 was initially examined because production of attenuated negative-stranded vaccine viruses by electroporation is ultimately desired.

Materials: Expression plasmids pCITE RSV N, pCITE RSV P, pCITE M2-1, and pCITE RSV L, and plasmids containing full-length RSV antigenomes rRSVC4G and rRSVΔM2-2 have been previously described (Jin et al., Virol. 251 (1998):206-214; Jin et al., J. Virol. 74 (2000):74-82). The plasmid containing the RSV antigenome rA2 cpts248/404/1030ΔSH was developed in the laboratory at the National Institute of Allergy and Infectious Disease, National Institutes of Health and Wyeth Vaccines (Pearl River, N.Y.) (see Karron et al., J. Inf. Dis. 191 (2005):1093-1104). The plasmid used to express phage T7 RNA polymerase, pADT7(N)ΔpT7, was adapted at MedImmune, Inc. from pADT7 that was kindly provided by Rob Brazas Briefly, plasmid pADT7 was modified to enhance a Kozak sequence and to remove the T7 promoter.

Serum-free Vero cells were adapted to growth without serum at MedImmune, Inc. (Mountain View, Calif.) and were used at no more than passage 152. The cells were maintained by growth at 37° C. in optiPRO supplemented with 2 mM L-glutamine (Gibco).

Preparation of cells for electroporation: The vero cells adapted to growth in serum-free medium were split 1:2 one day prior to transfection. The cells were 70% to 90% confluent at the time of electroporation.

Preparation of plasmid DNA for electroporation: Plasmid DNA was prepared using a Qiagen maxiprep kit (Qiagen) according to the manufacturer's instructions. The concentration of plasmid DNA in the final resuspension of each plasmid was between 0.5 μg/μL and 2 μg/μL.

Electroporation: The plasmids used for electroporation, at the ratio discussed in Example 1, were dispensed into a microfuge tube. 10⁷ serum-free Vero cells were added to the plasmids and the total volume was brought to 300 μL with Opti-C. After gentle mixing, the combined cells and DNA were transferred to a cuvette and inserted into a BioRad Gene Pulser X cell electroporator set to 220 V and 950 microfarads. After one pulse of current, the cells were transferred to a TC6-well plate containing 2 ml of SFM4 MegaVir and incubated overnight at 35° C. Seven to fourteen days later, fresh serum-free Vero cells were inoculated with both the cells and supernatant harvested from the transfection. Two to five such additional passages were made at 7-14 day intervals. For the third passage, duplicate wells in TC6-well plates were inoculated with the cells and supernatant of the second passage. Three to seven days after the third passage, one of the duplicate wells in the TC6-well plates was immunostained to confirm that virus had been recovered. Cells and supernatants from the second of the duplicate wells were collected and virus was titered. 7 to 17 transfections were performed in any one experiment and a total of 3 independent experiments were performed.

Immunostaining: Immunostaining to detect RSV was performed by fixing infected cell monolayers in TC6-well plates in methanol followed by addition of a goat polyclonal antibody directed to RSV (Chemicon) diluted 1:1000 in PBS containing 5% powdered milk (w/v) (PBS-milk). The cells were then incubated with an anti-goat antibody conjugated to horseradish peroxidase (HRP) (Dako) which had been diluted 1:1000. To visualize the infected cells 3-amino-9-ethylcarbazole (AEC) (Dako) was added. To detect hMPV, infected cell monolayers were immunostained using ferret polyclonal antisera directed to hMPV, followed by addition of HRP-conjugated goat anti-ferret antibody (Dako), and finally AEC. The ferret polyclonal antisera were generated by intranasally infecting 8-10 week old ferrets with 6.0 log₁₀ hMPV/NL/1/00 and collecting blood 4 weeks after infection.

Results: When 46 μg of total plasmid DNA was used, virus was recovered by passage 3 in 4 out of 12 transfections (Table 1). In contrast, no rRSVΔM2-2 virus was recovered when the total amount of DNA was 11.5 μg, 23 μg, 69 μg or 96 μg. 46 μg of total DNA corresponded to 8 μg, of pCITE RSV N, 8 μg pCITE RSV P, 6 μg of pCITE RSV M2-1, 4 μg of pCITE RSV L, 10 μg of rRSVΔM2-2 and 10 μg pADT7(N)ΔpT7.

TABLE 1
Recovery of virus using varying amounts of plasmid DNA

| | Total | | |
|---|---|---|---|
| | plasmid DNA | No. recoveries/ | |
| Virus | (μg) | No. attempts | % recovery |
| rRSVΔM2-2 | 11.5 | 0/3 | 0 |
| | 23 | 0/10 | 0 |
| | 46 | 4/12 | 33 |
| | 69 | 0/2 | 0 |
| | 92 | 0/2 | 0 |
| rhMPV/NL/E93K/S101P | 23 | 0/6 | 0 |
| | 46 | 9/10 | 90 |
| rRSVC4G | 46 | 4/6 | 67 |

*All plasmid DNA was prepared using bovine RNase and standard solutions supplied with Qiagen maxiprep kit.

### Example 3

### Forty-Six μg Total Plasmid DNA Used for Electroporation Rescued Additional Non-Segmented RNA Viruses

A high quantity of total DNA, 46 μg, was able to achieve recovery of rRSVΔM2-2 virus from serum-free cells with electroporation. To determine if another paramyxovirus could be rescued using 46 μg total DNA the same protocol as described Example 2 the same Vero cell preparation, plasmid preparation, and electroporation steps were repeated for rescue of a recombinant hMPV strain, rhMPV/NL/1/00/E93K/S101P, derived from rhMPV/NL/1/00. Rescue of rRSVC4G with 46 μg total DNA was attempted as well.

Materials: Plasmids used for rescue of hMPV included antigenomic plasmid rhMPV/NL/1/00/E93K/S101P, and expression plasmids pCITE hMPV N, pCITE hMPV P, pCITE hMPV M2-1, and pCITE hMPV L. These plasmids were described in Herfst S. et al., J. Virol. 78 (2004):8264-8270 and Schickli et al., J. Virol. 79 (2005):10678-10689. Plasmids used for rescue of rRSVC4G were described in Example 1.

Results: As observed with rRSVΔM2-2, virus was not recovered from cells transfected with 11.5 μg or 23 μg total DNA, but was recovered from cells electroporated with 46 μg total DNA. rhMPV/NL/1/00/E93K/S101P virus was recovered in 9 out of 10 attempts when a total of 46 μg DNA was used (See Table 1). rRSVC4G was recovered from cells in 4 of 6 electroporation attempts using the total of 46 μg DNA. Thus, efficiencies of virus recovery ranged from 33% to 90% in serum-free Vero cells using standard plasmid preparations.

### Example 4

### Effect of RNase Source Used During Plasmid Preparation on Recovery of Non-Segmented RNA Viruses from Electroporated Cells

The Qiagen maxiprep kit (Qiagen) used to prepare plasmid DNA for electroporation of cells in Examples 2 and 3 utilized a bovine RNase. An optimal system generating viruses for use in clinical studies would not utilize any animal-derived materials, including this bovine enzyme. Two alternatives were substituted for the bovine-derived RNase A treatment, (1) yeast-derived recombinant bovine RNase A treatment and (2) extensive DNA column washing (7 volumes (210 ml) of wash buffer were used in the wash step instead of 2 volumes (60 ml)).

Method: rRSVΔM2-2 was prepared by three methods: (1) as described in Example 2, (2) as described in Example 2 with the exception that 200 μg recombinant RNase A (Ambion) was added to the PI buffer supplied in the Qiagen maxiprep kit in place of bovine-derived RNase A, and (3) as described in Example 2 except that bovine RNase A was not added to the Qiagen PI buffer and the DNA column was washed with seven volumes of wash buffer instead of two at the wash step.

Results: Virus could not be recovered, 0 of 11 attempts, from cells electroporated with DNA prepared from Qiagen maxiprep kits in which the bovine RNase was substituted with the recombinant RNase A. See Table 2. These data suggest that a component of the recombinant RNase A preparation may interfere with the electroporation procedure. Virus could be recovered, in 1 of 7 attempts, from cells transfected with plasmid DNA that was prepared in the absence of bovine RNase A and that had been extensively washed. It should be noted that substituting excess washing steps for bovine RNase treatment did not completely remove RNA from the preparation (data not shown). Thus, failure to completely remove RNA impeded, but did not absolutely prevent, virus recovery.

TABLE 2
Recovery of virus using plasmid DNA prepared with
RNase from different sources or no RNase

| | | No. recoveries/ | |
|---|---|---|---|
| Virus | RNase | No. attempts | % Recovery |
| rRSVΔM2-2 | Bovine | 4/12 | 33 |
| | Recombinant | 0/11 | 0 |
| | none | 1/7 | 14 |

*All transfections were preformed using 46 μg total DNA and plasmid DNA was not made with endotoxin-free materials.

### Example 5

### Endotoxin Removal During Plasmid Preparation Improves Virus Recovery from Electroporated Cells

Method: Vero cells were prepared and electroporation was performed as described in Example 2. The plasmid DNA that was used for the electroporation of the Vero cells was prepared similarly to the description in Example 2 with the following exceptions: (1) endotoxins were removed from the preparation using a proprietary endotoxin removal reagent developed by Qiagen; or (2) endotoxins were removed from the preparation using the proprietary endotoxin removal reagent and bovine-derived RNase A treatment was omitted.

Results: High rates of recovery of both rRSVΔM2-2 and rRSVC4G were observed when cells were electroporated with endotoxin-free plasmid preparations. In the case of rRSVΔM2-2, virus was recovered in 90% of the electroporation attempts when plasmids were prepared using the endotoxin removal reagent and bovine-derived RNase A. Omission of bovine-derived RNase A in addition to use of the endotoxin removal reagent resulted in recovery of the rRSVΔM2-2 virus in 100% of the electroporation attempts. See Table 3. Similar rates of recovery were observed for recovery of rRSVC4G. See Table 3. These data demonstrated efficient recovery of non-segmented negative strand RNA viruses from electroporated cells in the absence of any animal derived products.

TABLE 3
Recovery of virus using plasmid DNA prepared with endotoxin
removal reagent and with or without bovine RNase

| | | Endotoxin-free | No. recoveries/ | % |
|---|---|---|---|---|
| Virus | RNase | plasmid DNA (μg) | No. attempts | recovery |
| rRSVΔM2-2 | Bovine | Yes | 9/10 | 90 |
| | Bovine | No | 4/12 | 33 |
| | None | Yes | 10/10 | 100 |
| | None | No | 1/7 | 14 |
| rRSVC4G | Bovine | Yes | 2/2 | 100 |
| | Bovine | No | 4/6 | 67 |
| | None | Yes | 3/3 | 100 |
| | None | No | Not done | |

*All transfections were performed with 46 μg of total plasmid DNA.

### Example 6

### Vero Cell Viability in Recovery Conditions for Temperature-Sensitive Viruses

While rRSVΔM2-2 and rRSVC4G could be efficiently recovered from electroporated cells in the absence of any animal or animal-derived products, temperature-sensitive (ts) RSV rA2 cpts248/404/1030ΔSH appeared to require serum. The Vero cells that had been electroporated for production of the ts RSV rA2 cpts248/404/1030ΔSH virus were grown at 32° C. following electroporation, the permissive temperature for replication of this ts virus. It was possible that the conditions under which the Vero cells were grown for recovery of the ts virus were detrimental to Vero cell growth.

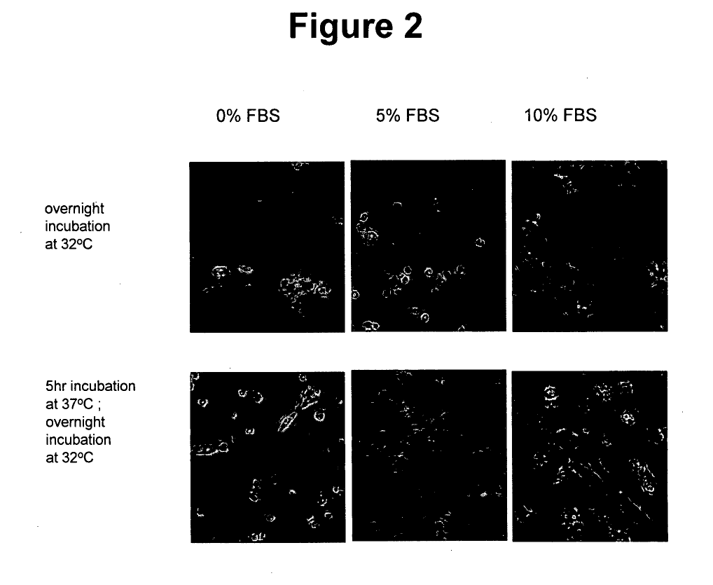

The effect of incubating Vero cells at 37° C. versus 32° C. and the effect of incubating Vero cells with or without FBS was investigated to determine Vero cell viability under these various conditions. To conduct this investigation, six groups of serum-free Vero cells were electroporated with 46 μg total plasmid (endotoxin-free and prepared in the absence of RNase A) and then treated as follows: (1) incubated overnight at 32° C. in serum-free medium; (2) incubated overnight at 32° C. in medium containing 5% FBS; (3) incubated overnight at 32° C. in medium containing 10% FBS; (4) incubated 5 hours at 37° C. before incubation at 32° C. overnight in serum-free medium; (5) incubated 5 hours at 37° C. before incubation at 32° C. overnight in medium containing 5% FBS; (6) incubated 5 hours at 37° C. before incubation at 32° C. overnight in medium containing 10% FBS. Incubation of the cells under each of these conditions revealed that Vero cell recovery following electroporation can be improved by incubation at 37° C. for 5 hours immediately following electroporation and by addition of FBS. See FIG. 2.

### Example 7

### Addition of Growth Factors to Electroporated Vero Cells Improves ts Virus Recovery

The addition of FBS to the Vero cells following electroporation likely provided unidentified growth factors that stimulate the recovery of cell membrane structures damaged during electroporation. It was found that Vero cell recovery following electroporation was improved by addition of any one of three recombinant growth factors: human epidermal growth factor (rEGF), human transforming growth factor alpha (rTGFA), and human insulin-like growth factor I (rIGF-I) (data not shown). To determine if the improved Vero cell recovery translated to improved generation of ts RSV rA2 cpts248/404/1030ΔSH, Vero cells were electroporated with plasmids pCITE RSV N, pCITE RSV P, pCITE M2-1, pCITE RSV L, and plasmids containing full-length RSV antigenome rA2 cpts248/404/1030ΔSH and plasmid encoding T7 RNA polymerase. Following electroporation 10 ng/ml rEGF, 20 ng/ml rTGFA, or 20 ng/ml rIGF-I was added to the culture medium. One day post-electroporation the cells were photographed and were then used to inoculate fresh serum-free Vero cells in each of duplicate TC6-well plates. Seven days later, one set of the duplicate TC6-wells were immunostained with polyclonal antibody specific for RSV. The cells in the second set of the duplicate TC6-wells were used to inoculate fresh Vero cells for a second passage.

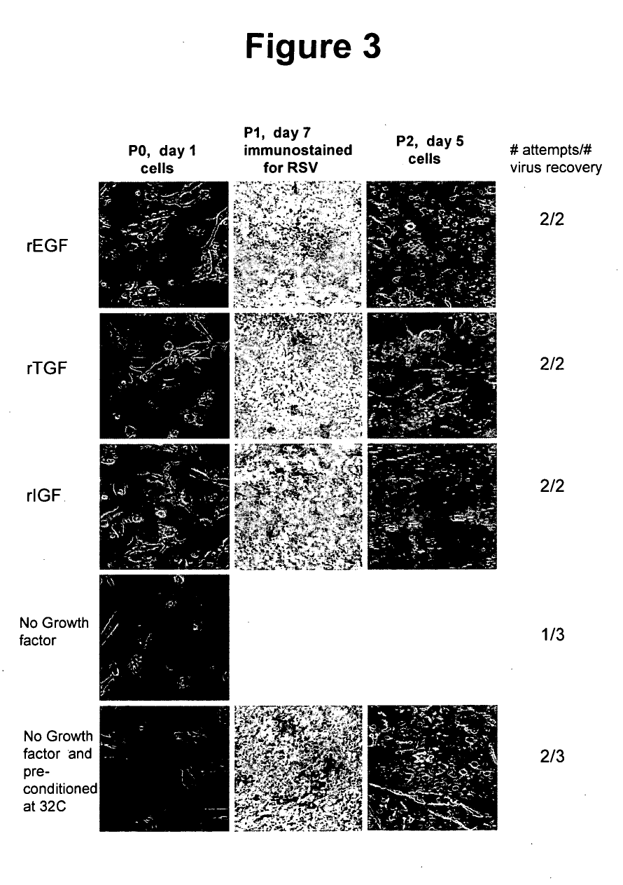

In the set of wells that were immunostained with RSV, it was observed that treatment with any of the three growth factors resulted in RSV production. See FIG. 3, which shows that 100% of wells containing Vero cells that received growth factor following electroporation produced the ts virus. Meanwhile, only a third of wells containing cells that did not receive growth factor following electroporation produced the ts virus.

In the set of wells that were used to inoculate fresh Vero cells for a second passage, five days following the second passage the cells were inspected for syncytia formation and were photographed. Wells of Vero cells that had received growth factor following electroporation showed syncytia formation. See FIG. 3. Thus, the addition of any of these recombinant growth factors permitted recovery of virus from Vero cells under temperatures permissive for ts virus replication and recovery.

### Example 8

### Pre-Conditioning Vero Cells Prior to Electroporation Improves ts Virus Recovery

Preconditioning Vero cells to the 32° C. post-electroporation temperature at which the recombinant ts RSV replicates was also tested for ability to improve virus recovery. Vero cells were grown for one passage at 32° C. and were then split so that they would be 70% to 90% confluent by the second passage. These cells were then used for electroporation. As can be seen in FIG. 3, rA2 cpts248/404/1030ΔSH was recovered from 2 of 3 electroporations performed with pre-conditioned Vero cells.

### Example 9

### A Human PIV3 Temperature-Sensitive Virus, rcp45 hPIV3 was Recovered in Serum-Free Vero Cells by Electroporation

In this example, an hPIV3 virus was recovered by electroporation using serum-free materials. The hPIV3 virus was temperature-sensitive vaccine candidate rcp45. It was recovered when 40 ug of total DNA was employed during transfection of 10⁷ serum-free Vero cells, and while using only serum-free reagents.

Materials: hPIV3 plasmids used for rescue of rcp45 hPIV3 included antigenomic full-length plasmid, and expression plasmids pCITE PIV3 N, pCITE PIV3 P, and pCITE PIV3 L. These plasmids were described in Skiadopoulos et al, Journal of Virology, 1999, 73:1374-1381.

Results: Virus was readily recovered when 40 ug of DNA was transfected into 10⁷ serum-free Vero cells, using only serum-free reagents. The 40 ug DNA was 8 ug pCITE PIV3 N, 8 ug pCITE PIV3 P, 4 ug pCITE PIV3 L, 10 ug full-length rcp45 hPIV antigenomic DNA, and 10 ug plasmid encoding T7 RNA polymerase. These quantities are the same quantities as used for recovery of RSV ΔM2-2, rA2 cpts 248/404/1030/ΔSH and hMPV/NL/E939K/S101P in the other examples above, except the pCITE M2-1 plasmid is not used for recovery of PIV3 viruses. The parameters for electroporation were the same 950 microfarads and 220 volts. The plasmids were all endotoxin-free and prepared without RNase A. The rcp45 virus is temperature-sensitive and virus recovery was conducted at 32° C. Virus was successfully recovered in 3 out of 5 electroporations.

1. A serum-free method of preparing a non-segmented negative-stranded RNA virus, comprising:
providing a host cell in a serum-free medium;
electroporating the host cell with nucleic acids, comprising:
one or more expression vectors encoding ribonucleoprotein complex proteins of the non-segmented negative-stranded RNA virus; and
a vector comprising a nucleotide sequence of a genome or antigenome of the non-segmented negative-stranded RNA virus; and
recovering the non-segmented negative-stranded RNA virus.

2. An animal protein-free method of preparing a non-segmented negative-stranded RNA virus, comprising:
providing a host cell in an animal protein-free medium;
electroporating the host cell with nucleic acids which are animal protein-free comprising:
one or more expression vectors encoding the ribonucleoprotein complex proteins of the non-segmented negative-stranded RNA virus; and
a vector comprising a nucleotide sequence of a genome or antigenome of the non-segmented negative-stranded RNA virus; and
recovering the non-segmented negative-stranded RNA virus.

3. The method of claims 1 or 2 wherein prior to the step of recovering, the electroporated host cells and/or media from the electroporated host cells are passaged on expansion cells.

4. The method of claims 1 or 2 wherein the non-segmented negative-stranded RNA virus is attenuated.

5. The method of claims 1 or 2 wherein the non-segmented negative-stranded RNA virus is a family paramyxoviridae virus.

6. The method of claim 5 wherein the family paramyxoviridae virus is a subfamily pneumovirinae virus.

7. The method of claim 6 wherein the subfamily pneumovirinae virus is respiratory syncytial virus.

8. The method of claim 7 wherein the respiratory syncytial virus does not express an M2-2 gene.

9. The method of claim 5 wherein the family paramyxoviridae virus is parainfluenza virus.

10. The method of claim 6 wherein the subfamily pneumovirinae virus is metapneumovirus.

11. The method of claim 1 or 2 wherein the electroporated host cells are further incubated in a medium comprising a growth factor.

12. The method of claim 11 wherein the growth factor is one of an epidermal growth factor, a transforming growth factor alpha, or an insulin-like growth factor.

13. The method of claims 1 or 2 wherein the one or more expression vectors and the vector comprising the nucleotide sequence of the genome or antigenome of the non-segmented negative-stranded RNA virus are plasmids.

14. The method of claim 13 wherein total quantity of plasmids is between about 40 and 50 μg per approximately 10⁶ to 10⁸ host cells.

15. The method of claims 1 or 2 wherein:
expression of the ribonucleoprotein complex proteins is under control of a promoter of a polymerase not natively expressed by the host cells, and
wherein the host cells are further electroporated with a further expression vector encoding the polymerase not natively expressed by the host cells.

16. The method of claim 15 wherein the polymerase is T7, SP6 or T3.

17. The method of claim 13 wherein the plasmids are endotoxin free.

18. The method of claims 1 or 2 wherein the host cells are Vero cells.

19. A serum-free method of preparing a pneumovirinae virus comprising:
providing a host cell in a serum-free medium;
electroporating host cells with nucleic acids comprising:
expression plasmids encoding N, P, and L proteins of the pneumovirinae virus, wherein expression of the N, P, and L proteins of the pneumovirinae virus are under control of a promoter for T7 polymerase;
an expression plasmid encoding T7 polymerase; and
a plasmid comprising a nucleotide sequence of a genome or
antigenome of the pneumovirinae virus;
culturing the electroporated host cells on expansion cells; and
recovering the pneumovirinae virus from the expansion cells cultured with the electroporated host cells; wherein the plasmids utilized in the step of electroporating are endotoxin-free.

20. The method of claim 19 wherein the pneumovirinae virus is a respiratory syncytial virus.

21. The method of claim 19 wherein the pneumovirinae virus is a metapneumovirus.

22. The method of claim 19 wherein the host cells are further electroporated with an expression plasmid encoding M2-1 under control of the promoter for T7 polymerase.

23. The method of claim 19 wherein the host cells are Vero cells.

24. The method of claim 19 wherein the expansion cells are Vero cells.

25. The method of claim 22 wherein the molar ratio of expression plasmids encoding the N protein, the P protein, the M2-1 protein, the L protein, the plasmid comprising the nucleotide sequence of the genome or antigenome, and the expression plasmid encoding the T7 polymerase is about 4:4:3:1:1.5:4.

26. The method of claim 25 wherein the total quantity of plasmids is between about 40 and 50 μg per approximately 10⁶ to 10⁸ host cells.

27. The method of claim 26 wherein the total quantity of plasmids is between about 45 and 50 μg.

28. The method of claim 19 wherein the electroporated host cells are further incubated in a medium comprising a growth factor.

29. The method of claim 19 wherein the method is further animal protein-free.

30. The method of claim 19 wherein the electroporated host cells and the electroporated host cells transferred to the culture of expansion cells are incubated at about 32° C.

31. The method of claim 19 wherein the non-segmented negative-stranded RNA virus is further attenuated.
